(19) United States
(12) Patent Application Publication
(10) Pub. No.: US 2026/0215807 A1
(43) Pub. Date: Jul. 30, 2026

(54) DEVICES, METHODS AND CATHETERS FOR TISSUE DAMAGE PROTECTION IN HIGHLY CALCIFIED VESSELS DURING ATHERECTOMY PROCEDURES

(71) Applicant: BARD PERIPHERAL VASCULAR, INC., Franklin Lakes, NJ (US)

(72) Inventors: Aseem SINGH, Tempe, AZ (US); Austin T. JONES, Mesa, AZ (US); Ikshwaku ATODARIA, Mesa, AZ (US)

(73) Assignee: BARD PERIPHERAL VASCULAR, INC., Franklin Lakes, NJ (US)

(21) Appl. No.: 19/151,929

(22) PCT Filed: Feb. 1, 2023
(86) PCT No.: PCT/US2023/061773

§ 371 Date: Jul. 29, 2025

(51) Int. Cl. A61B 17/3207; A61B 17/00

(52) U.S. Cl. CPC A61B 17/320758; A61B 2017/00075; A61B 2017/00398; A61B 2017/00778

(57) ABSTRACT

Devices and methods including a catheter, a mechanical cutting element coupled to a first end of the catheter, a drive motor coupled to a second end of the catheter, an audio sensor coupled to the catheter, and a processor coupled to the catheter. The processor is programmed to perform operations including receiving a set of signals from the audio sensor and the drive motor generated when the drive motor is activated, extracting a set of features from the set of signals, estimating a diameter of clearance of the mechanical cutting element based on the set of features, generating a control signal based on the diameter of clearance, and controlling the drive motor based on the control signal.

### TECHNICAL FIELD

The present disclosure relates to atherectomy catheters, and more particularly to preventing clinical complications during an atherectomy procedure.

### BACKGROUND

Atherectomy is a procedure to remove plaque from an artery, the cause of peripheral artery disease and coronary artery disease. Plaque or calcium is removed from the artery by shaving with rotating blades or burrs or vaporizing away with a laser on the end of a catheter inserted into the artery. Current atherectomy procedures may result in perforation of the arterial vessels due to the absence of any active feedback when the device is nearing the arterial wall. Additionally, the atherectomy device interaction with the plaque and/or artery during the procedure is limited to fluoroscopy/ultrasound imaging or physician experience.

Therefore, intelligent strategies for performing atherectomy procedures that can actively reduce the likelihood of or prevent clinical complications are desired.

### SUMMARY

In accordance with one embodiment of the present disclosure, a device includes a catheter, a mechanical cutting element coupled to a first end of the catheter, a drive motor coupled to a second end of the catheter, an audio sensor coupled to the catheter, and a processor coupled to the catheter. The processor is programmed to perform operations including receiving a set of signals from the audio sensor and the drive motor generated when the drive motor is activated, extracting a set of features from the set of signals, estimating a diameter of clearance of the mechanical cutting element based on the set of features, generating a control signal based on the diameter of clearance, and controlling the drive motor based on the control signal.

In accordance with another embodiment of the present disclosure, a method comprises receiving, from an audio sensor and a drive motor of a catheter, a set of signals generated when the drive motor is activated, extracting, with a processor of the catheter, a set of features from the set of signals, estimating a diameter of clearance of a mechanical cutting/grinding element of the catheter based on the set of features, generating a control signal based on the diameter of clearance, and controlling the drive motor based on the control signal.

In accordance with one other embodiment of the present disclosure, a catheter includes a mechanical cutting element coupled to a first end of the catheter, a drive motor coupled to a second end of the catheter, an audio sensor coupled to the catheter, and a processor coupled to the catheter. The processor is programmed to perform operations including receiving a set of signals from the audio sensor and the drive motor generated when the drive motor is activated, extracting a set of features from the set of signals, estimating a diameter of clearance of the mechanical cutting/grinding element based on the set of features, generating a control signal based on the diameter of clearance, and controlling the drive motor based on the control signal.

These and additional features provided by the embodiments described herein will be more fully understood in view of the following detailed description, in conjunction with the drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 depicts a computer-implemented system including modules for use with control schemes and process flows herein, according to one or more embodiments shown and described herein;

FIG. 2 depicts a device including a catheter for use with the system of FIG. 1, according to one or more embodiments shown and described herein;

FIG. 3 depicts a workflow for controlling the catheter of FIG. 2 using the system of FIG. 1, according to one or more embodiments shown and described herein;

FIG. 4 depicts a process flow for using the system of FIG. 1 and device of FIG. 2, according to one or more embodiments shown and described herein; and

FIG. 5 depicts a performance diagram of the device of FIG. 2, according to one or more embodiments shown and described herein.

### DETAILED DESCRIPTION

The embodiments disclosed herein include devices, methods, and catheters for tissue damage protection in highly calcified vessels during atherectomy procedures. In embodiments disclosed herein, tissue damage protection is enabled by monitoring audio and/or motor performance characteristics associated with an atherectomy catheter (also referable to herein as a catheter) to determine the type of lesion or substance with which the catheter has come into contact. The catheter may further be associated with a machine learning model that receives real-time audio and/or motor performance parameters from the catheter to determine the change in a substance with which the catheter is interacting to further estimate a diameter of clearance with respect to the substance and for the catheter. Doing so allows the catheter to minimize damage to healthy tissue without losing the ability to ablate lesions.

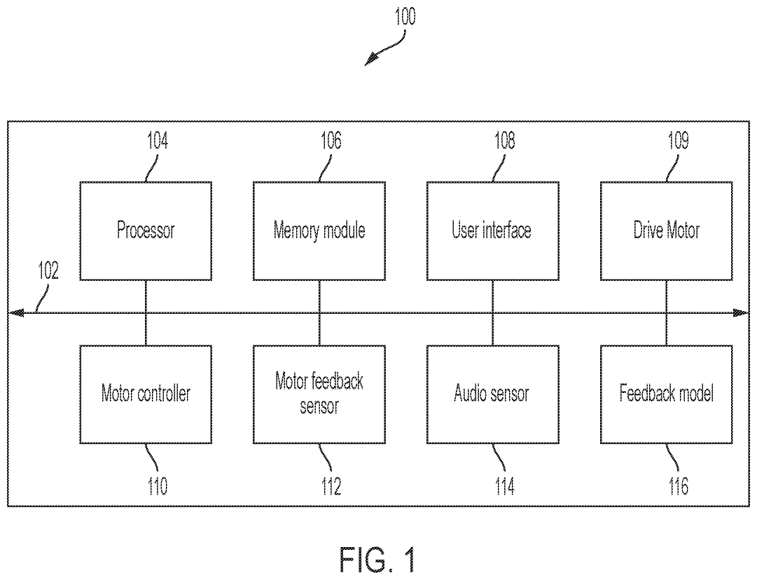

Referring now to FIG. 1, a system 100 as a computer-implemented system including modules for use with control schemes and process flows herein is depicted. The system 100 may be communicatively connected to a catheter to control the catheter for tissue damage protection. In some embodiments, one or more modules of the system 100 may be disposed in or remote from the catheter. The system 100 may include at least a processor 104, a memory module 106, a user interface 108, a drive motor 109, a motor controller 110, a motor feedback sensor 112, an audio sensor 114, and a feedback module 116. The system 100 may further include a communication path 102 for communicatively coupling the various components of the system 100.

The processor 104 may include one or more processors that may be any device capable of executing machine-readable and executable instructions. Accordingly, each of the one or more processors of the processor 104 may be a controller, an integrated circuit, a microchip, or any other computing device. The processor 104 is coupled to the communication path 102 that provides signal connectivity between the various components of the system 100. Accordingly, the communication path 102 may communicatively couple any number of processors of the processor 104 with one another and allow them to operate in a distributed computing environment. Specifically, each processor 104 may operate as a node that may send and/or receive data. As used herein, the phrase “communicatively coupled” means that coupled components are capable of exchanging data signals with one another such as, e.g., electrical signals via conductive medium, electromagnetic signals via IR, optical signals via optical waveguides, and the like.

The communication path 102 may be formed from any medium that is capable of transmitting a signal such as, e.g., conductive wires, conductive traces, optical waveguides, and the like. In some embodiments, the communication path 102 may facilitate the transmission of wireless signals, such as Wi-Fi, Bluetooth Near-Field Communication (NFC), and the like. Moreover, the communication path 102 may be formed from a combination of mediums capable of transmitting signals. In one embodiment, the communication path 102 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical, or electromagnetic), such as DC, AC, sinusoidal-wave, triangular-wave, square-wave, vibration, and the like, capable of traveling through a medium.

The memory module 106 is communicatively coupled to the communication path 102 and may contain one or more memory modules comprising RAM, ROM, flash memories, hard drives, or any device capable of storing machine-readable and executable instructions such that the machine-readable and executable instructions can be accessed by the processor 104. The machine-readable and executable instructions may comprise logic or algorithms written in any programming language of any generation (e.g., 1GL, 2GL, 3GL, 4GL, or 5GL) such as, e.g., machine language, that may be directly executed by the processor, or assembly language, object-oriented languages, scripting languages, microcode, and the like, that may be compiled or assembled into machine-readable and executable instructions and stored on the memory module 106. Alternatively, the machine-readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

The user interface 108 is coupled to the communication path 102 and may contain hardware for receiving input from a user. Hardware for receiving input may include devices that send information to the processor 104. For example, a keyboard, mouse, scanner, touchscreen, camera, dial, button, and the like are all user interface devices because they provide input to the processor 104 from the user. When a user interacts with a user interface 108, the interaction may be transmitted to the processor 104 via the communication path 102 for use by the processor 104. For example, and not as a limitation, the user interface 108 may include a dial that, when operated by the user, sends signals to the processor 104 and/or the motor controller 110 for controlling the speed of drive motor 109.

The motor feedback sensor 112 is coupled to the communication path 102 and communicatively coupled to the processor 104. The motor feedback sensor 112 may be one or more sensors coupled to a drive motor 109 for determining motion states, temperature states, electromagnetic states, and/or other features. Motion states may include position, speed, acceleration, mechanical strain, and/or the like. Temperature states may include temperature, head flow, and/or the like. Electromagnetic states may include voltage, current, charge, magnetic flux, pulse-width modulation (PWM), and/or the like. The motor feedback sensor 112 may also include mechanisms to filter noise from the sensed data. The drive motor 109 may be a brushless, brushed, induction, or any other kind of electric motor. Accordingly, the drive motor 109 may be battery-powered or otherwise connected to a power source. Alternatively, the drive motor 109 may be a turbine driven by compressed air or nitrogen. It should be understood that although discussion may be primarily held with regard to electric motors, other methods for controlling rotational speed are contemplated.

The audio sensor 114 is coupled to the communication path 102 and communicatively coupled to the processor 104. The audio sensor 114 may be one or more sensors coupled to the system 100 for determining volume, pitch, frequency, and/or features of sounds emitted from the motor or materials in contact with the motor. The audio sensor 114 may include a microphone or an array of microphones that may include mechanisms to filter background noise, such as beamforming.

The feedback module 116 may be a hardware module coupled to the communication path 102 and communicatively coupled to the processor 104. The feedback module 116 may also or instead be a set of instructions contained in the memory module 106. The feedback module 116 may be configured to receive signals from the motor feedback sensor 112 and/or audio characteristics from the audio sensor 114. The feedback module 116 may be further configured to train and utilize a machine learning model for generating clearance estimations between the catheter and arterial lesions and/or for classifying an arterial lesion that the catheter has come into contact with. The feedback module 116 may utilize supervised methods to train a machine learning model based on labeled training sets, wherein the machine learning model is a decision tree, a Bayes classifier, a support vector machine, a convolutional neural network, and/or the like. In some embodiments, unsupervised machine learning algorithms may be used, such as k-means clustering, hierarchical clustering, and/or the like. The feedback module 116 may also be configured to perform the methods as described herein.

It should be understood that the components illustrated in FIG. 1 are illustrative and are not intended to limit the scope of the present disclosure. More specifically, while the components in FIG. 1 are illustrated as residing within the system 100, this is a non-limiting example. In some embodiments, one or more of the components may reside external to system 100. It should be also be understood that the components of the system 100 described herein are exemplary and may contain more or less than the number of components shown in FIG. 1.

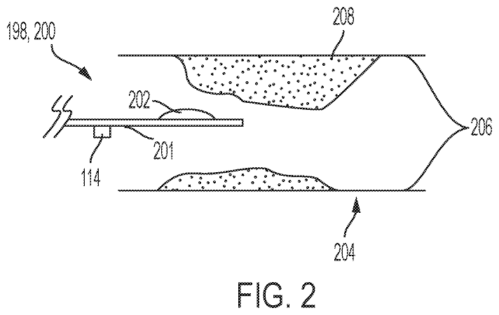

Referring now to FIG. 2, a catheter 200 of the system 100 is depicted, in which the catheter 200 is communicatively coupled to the system 100 via a device 198. Thus, the device 198 may comprise components of the catheter 200 and the system 100 as described herein. A body 201 of the catheter 200 may be in a tubular form. The body 201 may be made of a flexible, non-conductive material, such as silicone, polyurethane, and/or the like. The catheter 200 may have a first end. The first end of the catheter 200 may have a mechanical cutting element 202 attached thereto. In embodiments, the mechanical cutting element 202 may be configured to cut, grind, or otherwise remove lesions 208 from an artery 206. The second end of the catheter 200 may have a drive motor 109 (FIG. 1) coupled thereto as well as to the system 100, which may be each or collectively enclosed in a handle or other housing. In embodiments, the first end of the catheter 200 may be a distal end, and the second end may be a proximal end. The body 201 may be hollow thereby defining a cavity where elements connecting the drive motor 109 and/or mechanical cutting element 202 may be positioned. In some embodiments, other connective elements may be placed in the cavity. By way of example, and not as a limitation, if the audio sensor 114 is placed towards the first end of the catheter 200, then the communication path 102 may extend through the catheter 200 to connect the system 100 to the audio sensor 114. The body 201 of the catheter 200 may also include an insulation layer on the inside of the body 201 to protect elements within the body 201.

As previously stated, the first end of the catheter 200 may have a mechanical cutting element 202 attached thereto. The mechanical cutting element 202 may include one or more blades, burrs, beads, and/or any other element configured for shaving, cutting, sanding, grinding, or otherwise removing lesions 208 from an artery 206. As a non-limiting example, a mechanical cutting element 202 comprising one or more blades may be suitable for shaving and/or cutting lesions 208 as each blade contains a sharp edge suitable for cutting and/or grinding. Additionally or alternatively, a mechanical cutting element 202 comprising a burr may be suitable for sanding as the burr may be coated with diamond crystals on a leading edge. The lesions 208 in an artery may include plaque, calcium, and/or other build-ups in the artery 206 that obstructs blood flow within the artery 206.

The audio sensor 114 may be positioned at or near the first end of the catheter 200 where audio (such as acoustic waves based on the lesions 208 and/or artery 206 walls) may be the strongest. In some embodiments, the audio sensor 114 may be positioned at or near the second end of the catheter 200; for example, in a housing of the system 100. Although the second end of the catheter 200 is external to the atherectomy subject, plenty of high-pitched sounds can be heard of the subject's body and thus can be captured by the audio sensor 114. Positioning the audio sensor 114 at or near the second end of the catheter 200 may eliminate the issues around protecting and powering the audio sensor 114 as well as maintaining a connection with the audio sensor 114 to transmit information to the processor 104.

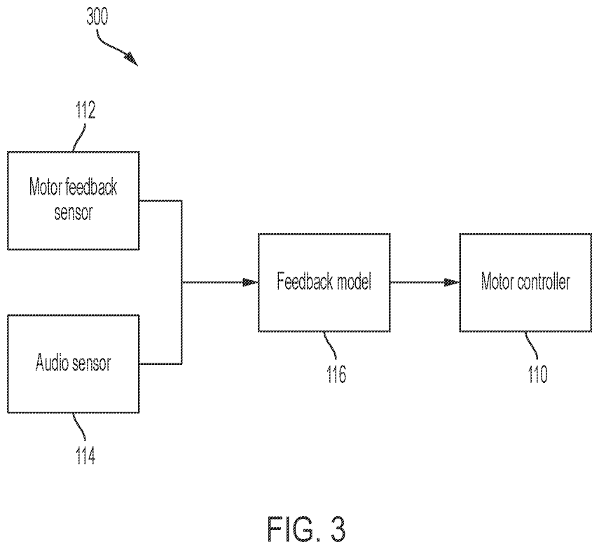

Referring now to FIG. 3, a control workflow 300 for controlling the catheter 200 using the system 100 is depicted. To prevent the mechanical cutting element 202 from causing damage to the wall 204 of the artery 206, the system 100 may determine a diameter between the mechanical cutting element 202 and a lesion 208 and/or a vessel wall 204, such as whichever may be nearer to the mechanical cutting element 202.

The feedback module 116 contains a machine learning model that may be trained prior to use. The machine learning model of the feedback module 116 may be trained to determine a type of substance (such as type of lesion) a mechanical cutting element 202 is interacting with based on a training data set. The training data set may comprise a set of features derived from a set of training signals generated by the audio sensor 114 and the motor feedback sensor 112 and labeled with a corresponding type of substance that a mechanical cutting/grinding element (e.g., the mechanical cutting element 202) was interacting with during the generation of the training signals. The machine learning model may also or instead be trained to estimate the diameter of clearance between the mechanical cutting element 202 and a surrounding lesion 208 and/or a vessel wall 204 based on a training data set comprising a set of features derived from a set of training signals generated by the audio sensor 114 and the motor feedback sensor 112 and labeled, such as manually, with a corresponding diameter of clearance around the mechanical cutting element 202. Features of the signals may include an amount, degree, amplitude, or any other characteristic of a signal. The features may be encoded in a standardized format suitable for training a machine learning model as an artificial intelligence component, such as one utilizing a neural network as described herein.

The motor feedback sensor 112 and/or the audio sensor 114 may send signals to the feedback module 116 and use the trained machine learning model. The feedback module 116 may extract features from the signals, encode the signals, and/or otherwise preprocess the signals before using the signals or derivatives thereof as inputs to the machine learning model. The machine learning model is configured to estimate the diameter of clearance of the mechanical cutting element 202 based on the set of features of the signals input to the feedback module 116. The machine learning model of the feedback module 116 may analyze the features extracted from the set of signals generated by the motor feedback sensor 112 and/or the audio sensor 114 to classify the set of signals as belonging to a particular level clearance within a blood vessel and/or to classify the signals as resulting from the interaction between the mechanical cutting element 202 and a type of substance. Knowing the type of substance that interacts with the mechanical cutting element 202 may help the feedback module 116 determine the amount of clearance that the mechanical cutting element 202 has with the vessel wall 204.

The feedback module 116 may output, via the machine learning model, an estimated diameter of clearance of the mechanical cutting element 202. The estimated diameter may be the smallest value of clearance, an average value of clearance, a value in a particular direction, and/or the like around the mechanical cutting element 202. The feedback module 116 may also output an estimated substance that the mechanical cutting element 202 is in contact with via the machine learning model.

The motor controller 110 may generate a control signal based on outputs from the feedback module 116. The control signal may be used to direct the drive motor 109 and in turn the mechanical cutting element 202 to perform ablation as appropriate and avoid unnecessary damage to healthy tissue. For example, the motor controller 110 may direct the drive motor 109 to increase speed in response to the feedback module 116 identifying an amount of clearance from the vessel wall 204 above a threshold level and/or identifying a hard substance such as a lesion 208. As another example, the motor controller 110 may direct the drive motor 109 to shut down in response to the feedback module 116 identifying an amount of clearance from the vessel wall 204 below the threshold level.

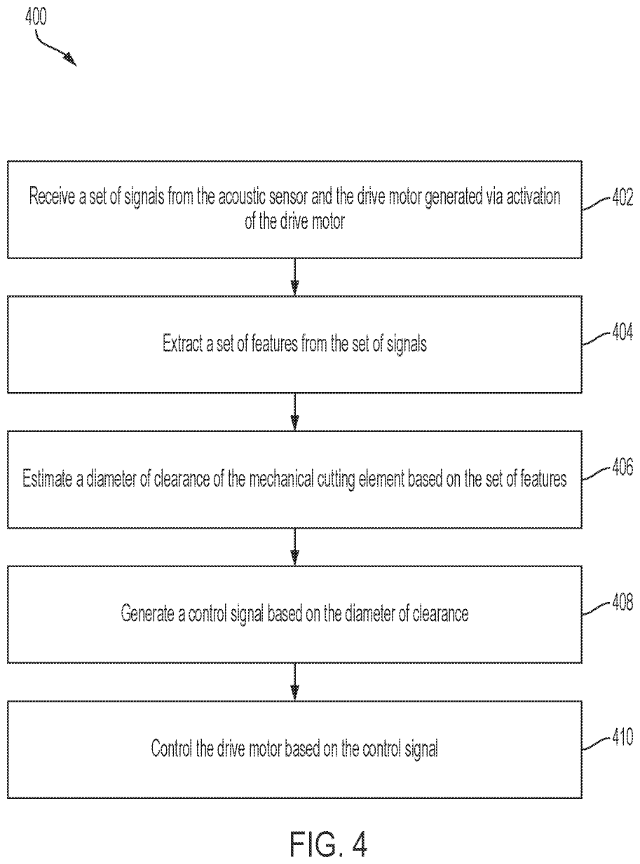

Referring now to FIG. 4, a flowchart of a method 400 is depicted. The method 400 may be carried out by a device such as the system 100. The method 400 is not limited to the steps or order of the steps as shown. An objective of the method 400 may be to prevent the mechanical cutting element 202 of the catheter 200 from causing damage to the wall 204 of an artery 206. In block 402, the system 100 receives a set of signals from the audio sensor 114 and the drive motor 109 via the motor feedback sensor 112. The set of signals is generated when the drive motor 109 is activated. The set of signals from the audio sensor 114 includes at least an audio signal of the drive motor 109, and the set of signals from the motor feedback sensor 112 includes at least current, voltage, torque, and/or speed of the drive motor 109. In some embodiments, the set of signals may be preprocessed with a noise reduction technique, such as filters or limiters.

In embodiments utilizing a supervised machine learning model, the machine learning model of the feedback module 116 may be trained before receiving the set of signals. The machine learning model of the feedback module 116 may be trained to determine a type of substance based on a training data set comprising a set of training features derived from a set of training signals generated by audio sensors 114 and motor feedback sensors 112 and labeled with a corresponding type of lesion. The set of training features may be the type of features to be discussed further with regard to block 404 below. Additionally or alternatively, the machine learning model may be trained to estimate the diameter of clearance between the mechanical cutting element 202 and a surrounding lesion 208 or a vessel wall 204 based on a training data set comprising a set of training features derived from a set of training signals generated by audio sensors 114 and motor feedback sensors 112 and labeled with a corresponding diameter of clearance.

In block 404, the system 100 extracts a set of features from the set of signals. The set of signals may be segmented into signals from the motor feedback sensor 112 and the audio sensor 114. The set of signals from the motor feedback sensor 112 may include motion states, temperature states, electromagnetic states, and/or other features. Motion states may include position, speed (RPM), acceleration, mechanical strain, and/or the like. Temperature states may include temperature, heat flow, and/or the like. Electromagnetic states may include voltage, current, charge, magnetic flux, pulse-width modulation (PWM), and/or the like. The motor feedback sensor 112 may also include mechanisms to filter noise from the sensed data. The set of signals from the audio sensor 114 may include a volume, pitch, frequency, and/or features of sounds emitted from the motor or substances interacting with the motor (e.g., lesion 208). Features of the signals may include, and not be limited to, an amount, degree, amplitude, and/or any other signal characteristic. The features may be encoded in a standardized format suitable for training a machine learning model.

In embodiments utilizing a supervised machine learning model, the machine learning model of the feedback module 116 may be trained before receiving the set of signals. The machine learning model may be trained based on a training data set comprising a set of training features derived from a set of training signals and labeled with a corresponding type of lesion or corresponding diameter of clearance of the mechanical cutting element 202. For example, the set of features extracted from training signals may include current. When the mechanical cutting element 202 encounters a graphite lesion, for example, the current may increase to maintain its speed while ablating the lesion.

In block 406, the system 100 estimates a diameter of clearance of the mechanical cutting element 202 based on the set of features. The machine learning model of the feedback module 116 may be a classifier that engages in unsupervised machine learning algorithms, such as k-means clustering, hierarchical clustering, and/or the like. The machine learning model of the feedback module 116 may analyze the features extracted from the set of signals generated by the motor feedback sensor 112 and/or the audio sensor 114 to classify the set of signals as belonging to a particular level clearance within a blood vessel. As a non-limiting example, if the features of a set of signals are similar to the set of training signals a clearance of 0.5 mm from vessel wall while ablating a calcium segment, the set of signals may be classified as indicating a clearance of 0.5 mm from the vessel wall.

The data collected by the system 100 may also be used to classify the type of substance encountered by the mechanical cutting element 202 as it advances through the vasculature during an atherectomy procedure. For example, the pitch of the sound may be the drive motor 109 and/or the mechanical cutting element 202 may change based on what it is moving through (e.g., lesions, plaques, free movement, etc.). Knowing or determining the type of substance that is encountered by the mechanical cutting element 202 may help the feedback module 116 determine the amount of clearance that the mechanical cutting element 202 has with the vessel wall 204. Audio features (e.g., volume or pitch) may respond by increasing to a degree as it approaches a vessel wall 204 when the mechanical cutting element 202 is in free movement. The audio features may further respond by increasing to a less significant degree as it approaches a vessel wall 204 when the mechanical cutting element 202 is ablating a lesion 208 than when in free, non-ablating movement.

In some embodiments, the system 100 may determine a diameter of clearance between the mechanical cutting element 202 and any neighboring substance and classify the type of substance encountered by the mechanical cutting element 202 in response to estimating the diameter of clearance to be zero. In such an embodiment, the feedback module 116 may contain multiple machine learning models including at least one for determining a diameter of clearance and one for classifying the type of substance encountered. In aspects, such multiple machine learning models are combinable into a single model. The feedback module 116 may perform supervised learning and/or unsupervised learning to appropriately determine a diameter of clearance and classify the type of substance encountered.

In block 408, the system 100 generates a control signal based on the diameter of clearance. The feedback module 116 may output, via the machine learning model, an estimated diameter of clearance of the mechanical cutting element 202. The estimated diameter may be the smallest value of clearance around the mechanical cutting element 202. The feedback module 116 may also output, via the machine learning model, an estimated substance that the mechanical cutting element 202 is in contact with. The processor 104 may generate a control signal based on outputs from the feedback module 116. The control signal may be used to direct the motor controller 110 and in turn the mechanical cutting element 202 to perform ablation as appropriate and avoid unnecessary damage to healthy tissue. For example, the control signal may cause the drive motor 109 to modify a speed of the mechanical cutting element 202 based on the diameter of clearance, such as reducing the speed in response to the diameter of clearance being below a threshold level. As another example, the control signal directs the drive motor 109 to shut down in response to contacting the vessel wall 204.

In block 410, the system 100 controls the drive motor 109 based on the control signal. The drive motor 109 may be controlled by the motor controller 110. The motor controller 110 may respond to the control signal immediately. The control signal may be overridden by a user input via the user interface 108.

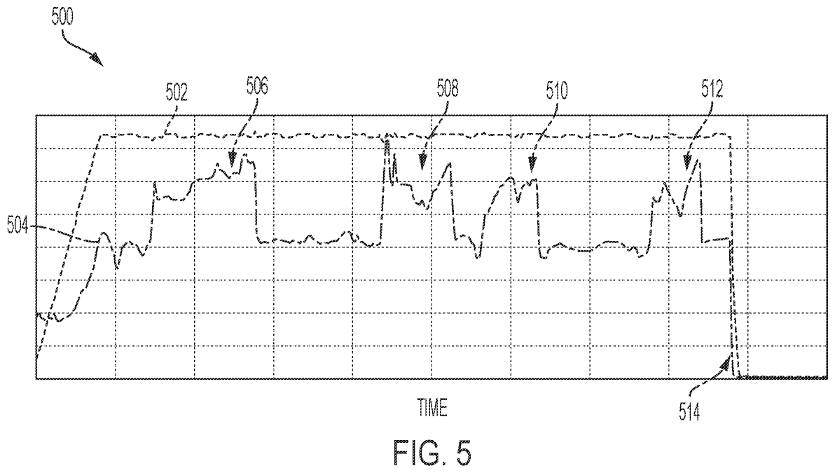

Referring now to FIG. 5, a performance diagram 500 of the catheter 200 is depicted. The performance diagram 500 may represent the speed 502 and current 504 of the drive motor 109. The system 100 may use the feedback from the interaction of the mechanical cutting element 202 with a substance, such as healthy tissue or lesion 208, to control the drive motor 109, such as speed 502 and torque. When the mechanical cutting element 202 (e.g., an orbital atherectomy burr) interacts with a lesion 208, the resistance is translated to the drive motor 109. The resistance requires more torque output from the drive motor 109 to maintain the same speed 502. The drive motor 109, in turn, draws more current 504 to deliver the determined torque.

The performance diagram 500 represents a time period when the mechanical cutting element 202 of the catheter 200 of the device 198 encounters a graphite lesion. Before the interaction occurs, the feedback module 116 may determine that the mechanical cutting element 202 is in free movement based on the minimal current 504 usage by the drive motor 109. When the current 504 picks up to two units above the X-axis of the performance diagram 500, the speed 502 of the drive motor 109 increases so that the mechanical cutting element 202 may begin ablation as the feedback module 116 recognizes the level of current 504 drawn by the drive motor 109 is indicative of previous instances of graphite lesions. As the mechanical cutting element 202 proceeds into the graphite lesion, the current 504 increases to maintain the speed 502 of the drive motor 109 at point 506.

Determining when to increase the speed of the mechanical cutting element 202 can allow the system 100 to keep the speed low to prevent unintended injury to healthy tissue until lesions or other unwanted substances are detected by the feedback module 116. In addition, some mechanical cutting elements 202, such as orbital atherectomy beads, have lower orbit diameters at lower speeds, which increases the amount of clearance of the mechanical cutting element 202. A lower orbit diameter would also reduce unintentional contact with healthy tissues, such as vessel walls 204. Furthermore, running at lower speeds 502 in when the mechanical cutting element 202 is in free movement also reduces the kinetic energy of the mechanical cutting element 202, thereby lowering the ability of the mechanical cutting element 202 to cause damage if the mechanical cutting element 202 were to come into contact with healthy tissue.

As the mechanical cutting element 202 proceeds through the graphite lesion at point 506, the mechanical cutting element 202 may encounter varying levels of density in the lesion 208. The current 504 drawn by the drive motor 109 to keep the mechanical cutting element 202 rotating at the same speed 502 may be reduced at less dense portions of the lesion 208 and be increased at more dense portions of the lesion 208, such as at points 508, 510, 512. While the system 100 monitors current 504 draw to maintain a low speed 502 until a lesion 208 is identified, the system 100 may also receive signals from the audio sensor 114. In some embodiments, the audio sensor 114 may provide additional information that the feedback module 116 may use, with or without information from the motor feedback sensor 112, to determine an amount of clearance of the mechanical cutting element 202. If the feedback module 116 determines that the diameter of clearance is below a threshold amount, the motor controller 110 may send a control signal to the drive motor 109 shut down. In which case, the speed 502 and current 504 of the drive motor 109 may be immediately reduced to zero, as shown at point 514. It should be understood that the example provided with regard to FIG. 5 is intended to be illustrative and non-limiting.

Embodiments may be further described with respect to the following numbered clauses:

1. A device comprising: a catheter; a mechanical cutting element coupled to a first end of the catheter; a drive motor coupled to a second end of the catheter; an audio sensor coupled to the catheter; and a processor coupled to the catheter and programmed to perform operations comprising: receiving a set of signals from the audio sensor and the drive motor generated when the drive motor is activated; extracting a set of features from the set of signals; estimating a diameter of clearance of the mechanical cutting element based on the set of features; generating a control signal based on the diameter of clearance; and controlling the drive motor based on the control signal.

2 The device of clause 1, wherein the audio sensor is coupled to the first end of the catheter.

3. The device of any preceding clause, wherein the set of signals from the audio sensor includes an audio signal and the set of signals from the drive motor includes at least one of a current, a voltage, a torque, and a speed.

4. The device of any preceding clause, wherein the processor is programmed to perform operations further comprising preprocessing the set of signals with a noise reduction technique.

5. The device of any preceding clause, wherein the processor is programmed to perform operations further comprising, before receiving the set of signals, training a machine learning model to estimate the diameter of clearance based on a training data set comprising a set of training features derived from a set of training signals generated by audio sensors and drive motors and manually labeled with a corresponding diameter of clearance.

6. The device of clause 5, wherein estimating the diameter of clearance of the mechanical cutting element comprises: inputting the set of features from the set of signals into the trained machine learning model; and generating the diameter of clearance of the mechanical cutting element with the trained machine learning model.

7. The device of clause 6, wherein the control signal directs the drive motor to modify a speed of the mechanical cutting element based on the diameter of clearance, in response to the diameter of clearance being below a threshold level.

8 The device of any preceding clause, wherein the processor is programmed to perform operations further comprising, before receiving the set of signals, training a machine learning model to determine a type of substance based on a training data set comprising a set of training features derived from a set of training signals generated by audio sensors and drive motors and manually labeled with a corresponding type of substance.

9 The device of clause 8, wherein the processor is programmed to perform operations further comprising: in response to estimating the diameter of clearance to be zero, inputting the set of features from the set of signals into the trained machine learning model; and generating the type of substance with the trained machine learning model.

10. The device of clause 9, wherein the control signal directs the drive motor to shut down in response to the type of substance being a vessel wall.

11. A method comprising: receiving, from an audio sensor and a drive motor of a catheter, a set of signals generated when the drive motor is activated; extracting, with a processor of the catheter, a set of features from the set of signals; estimating a diameter of clearance of a mechanical cutting element of the catheter based on the set of features; generating a control signal based on the diameter of clearance; and controlling the drive motor based on the control signal.

12. The method of clause 11, wherein the set of signals from the audio sensor includes an audio signal and the set of signals from the drive motor includes at least one of a current, a voltage, a torque, and a speed.

13. The method of any of clauses 11-12, further comprising preprocessing the set of signals with a noise reduction technique.

14. The method of any of clauses 11-13, further comprising, before receiving the set of signals, training a machine learning model to estimate the diameter of clearance based on a training data set comprising a set of training features derived from a set of training signals generated by audio sensors and drive motors and manually labeled with a corresponding diameter of clearance.

15. The method of clause 14, wherein estimating the diameter of clearance of the mechanical cutting element comprises: inputting the set of features from the set of signals into the trained machine learning model; and generating the diameter of clearance of the mechanical cutting element with the trained machine learning model.

16. The method of clause 15, wherein the control signal directs the drive motor to modify a speed of the mechanical cutting element based on the diameter of clearance, in response to the diameter of clearance being below a threshold level.

17. The method of any of clauses 11-17, further comprising, before receiving the set of signals, training a machine learning model to determine a type of substance based on a training data set comprising a set of training features derived from a set of training signals generated by audio sensors and drive motors and manually labeled with a corresponding type of substance.

18. The method of clause 17, further comprising: in response to estimating the diameter of clearance to be zero, inputting the set of features from the set of signals into the trained machine learning model; and generating the type of substance with the trained machine learning model.

19. The method of clause 18, wherein the control signal directs the drive motor to shut down in response to the type of substance being a vessel wall.

20. A catheter, comprising: a mechanical cutting element coupled to a first end of the catheter; a drive motor coupled to a second end of the catheter; an audio sensor coupled to the catheter; and a processor coupled to the catheter programmed to perform operations comprising: receiving a set of signals from the audio sensor and the drive motor generated when the drive motor is activated; extracting a set of features from the set of signals; estimating a diameter of clearance of the mechanical cutting element based on the set of features; generating a control signal based on the diameter of clearance; and controlling the drive motor based on the control signal.

It should now be understood that embodiments include devices, methods, and catheters for tissue damage protection in calcified vessels during atherectomy procedures. In embodiments, tissue damage protection is enabled by monitoring audio and/or drive motor performance to determine the type of lesion or substance with which an atherectomy catheter is interacting. The catheter may be equipped with a machine learning model that receives real-time audio and/or motor performance parameters to determine the change in a substance with which the catheter is interacting as well as an amount of clearance from the catheter to the substance and/or the vessel wall. Doing so allows the catheter to minimize damage to healthy tissue without losing the ability to ablate lesions.

For the purposes of describing and defining the present disclosure, it is noted that reference herein to a variable being a “function” of a parameter or another variable is not intended to denote that the variable is exclusively a function of the listed parameter or variable. Rather, reference herein to a variable that is a “function” of a listed parameter is intended to be open-ended such that the variable may be a function of a single parameter or a plurality of parameters.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

What is claimed is:

1. A device comprising:
a catheter;
a mechanical cutting element coupled to a first end of the catheter;
a drive motor coupled to a second end of the catheter;
an audio sensor coupled to the catheter; and
a processor coupled to the catheter and programmed to perform operations comprising:
receiving a set of signals from the audio sensor and the drive motor generated when the drive motor is activated;
extracting a set of features from the set of signals;
estimating a diameter of clearance of the mechanical cutting element based on the set of features;
generating a control signal based on the diameter of clearance; and
controlling the drive motor based on the control signal.

2. The device of claim 1, wherein the audio sensor is coupled to the first end of the catheter.

3. The device of claim 1, wherein the set of signals from the audio sensor includes an audio signal and the set of signals from the drive motor includes at least one of a current, a voltage, a torque, and a speed.

4. The device of claim 1, wherein the processor is programmed to perform operations further comprising preprocessing the set of signals with a noise reduction technique.

5. The device of claim 1, wherein the processor is programmed to perform operations further comprising, before receiving the set of signals, training a machine learning model to estimate the diameter of clearance based on a training data set comprising a set of training features derived from a set of training signals generated by audio sensors and drive motors and manually labeled with a corresponding diameter of clearance.

6. The device of claim 5, wherein estimating the diameter of clearance of the mechanical cutting element comprises:
inputting the set of features from the set of signals into the trained machine learning model; and
generating the diameter of clearance of the mechanical cutting element with the trained machine learning model.

7. The device of claim 6, wherein the control signal directs the drive motor to modify a speed of the mechanical cutting element based on the diameter of clearance, in response to the diameter of clearance being below a threshold level.

8. The device of claim 1, wherein the processor is programmed to perform operations further comprising, before receiving the set of signals, training a machine learning model to determine a type of substance based on a training data set comprising a set of training features derived from a set of training signals generated by audio sensors and drive motors and manually labeled with a corresponding type of substance.

9. The device of claim 8, wherein the processor is programmed to perform operations further comprising:
in response to estimating the diameter of clearance to be zero, inputting the set of features from the set of signals into the trained machine learning model; and
generating the type of substance with the trained machine learning model.

10. The device of claim 9, wherein the control signal directs the drive motor to shut down in response to the type of substance being a vessel wall.

11. A method comprising:
receiving, from an audio sensor and a drive motor of a catheter, a set of signals generated when the drive motor is activated;
extracting, with a processor of the catheter, a set of features from the set of signals;
estimating a diameter of clearance of a mechanical cutting element of the catheter based on the set of features;
generating a control signal based on the diameter of clearance; and
controlling the drive motor based on the control signal.

12. The method of claim 11, wherein the set of signals from the audio sensor includes an audio signal and the set of signals from the drive motor includes at least one of a current, a voltage, a torque, and a speed.

13. The method of claim 11, further comprising preprocessing the set of signals with a noise reduction technique.

14. The method of claim 11, further comprising, before receiving the set of signals, training a machine learning model to estimate the diameter of clearance based on a training data set comprising a set of training features derived from a set of training signals generated by audio sensors and drive motors and manually labeled with a corresponding diameter of clearance.

15. The method of claim 14, wherein estimating the diameter of clearance of the mechanical cutting element comprises:
inputting the set of features from the set of signals into the trained machine learning model; and
generating the diameter of clearance of the mechanical cutting element with the trained machine learning model.

16. The method of claim 15, wherein the control signal directs the drive motor to modify a speed of the mechanical cutting element based on the diameter of clearance, in response to the diameter of clearance being below a threshold level.

17. The method of claim 11, further comprising, before receiving the set of signals, training a machine learning model to determine a type of substance based on a training data set comprising a set of training features derived from a set of training signals generated by audio sensors and drive motors and manually labeled with a corresponding type of substance.

18. The method of claim 17, further comprising:
in response to estimating the diameter of clearance to be zero, inputting the set of features from the set of signals into the trained machine learning model; and
generating the type of substance with the trained machine learning model.

19. The method of claim 18, wherein the control signal directs the drive motor to shut down in response to the type of substance being a vessel wall.

20. A catheter, comprising:
a mechanical cutting element coupled to a first end of the catheter;
a drive motor coupled to a second end of the catheter;
an audio sensor coupled to the catheter; and
a processor coupled to the catheter programmed to perform operations comprising:
receiving a set of signals from the audio sensor and the drive motor generated when the drive motor is activated;
extracting a set of features from the set of signals;
estimating a diameter of clearance of the mechanical cutting element based on the set of features;
generating a control signal based on the diameter of clearance; and
controlling the drive motor based on the control signal.
